(19) United States
(12) Patent Application Publication
(10) Pub. No.: US 2025/0368664 A1
(43) Pub. Date: Dec. 4, 2025

(54) SURFACTANTS HAVING PEFA COMPOUNDS AND METHODS OF USE THEREOF

(71) Applicant: Ruby Bio Inc., San Carlos, CA (US)

(72) Inventors: Pavan Kumar Reddy Kambam, Sunnyvale, CA (US); Denis Kembero Ontumi, Dublin, CA (US); Charles Scott Silver, Oakland, CA (US)

(21) Appl. No.: 18/869,001

(22) PCT Filed: May 26, 2023
(86) PCT No.: PCT/US2023/067577

§ 371 Date: Nov. 25, 2024

Related U.S. Application Data
(60) Provisional application No. 63/365,404, filed on May 26, 2022.
(60) Provisional application No. 63/365,405, filed on May 26, 2022.
(60) Provisional application No. 63/365,406, filed on May 26, 2022.

(51) Int. Cl. C07F 9/141; A61K 8/37; A61K 8/55; A61Q 19/00; C07C 69/675; C11D 1/16; C11D 1/34; C11D 1/66

(52) U.S. Cl. CPC C07F 9/1411; A61K 8/375; A61K 8/55; A61Q 19/00; C07C 69/675; C11D 1/16; C11D 1/345; C11D 1/667

(57) ABSTRACT

Polyol esters of fatty acids (PEFAs) and their use as surfactants, methods of preparing, and compositions thereof are disclosed herein.

### REFERENCE TO RELATED APPLICATIONS

This application claims priority to U.S. Provisional Patent Application Ser. No. 63/365,404, entitled Compounds, Compositions, and Methods of Use Thereof, filed May 26, 2022; U.S. Provisional Patent Application Ser. No. 63/365,405, entitled Polyol Ester of Fatty Acids (PEFAs) as Antifoam, filed May 26, 2022; and U.S. Provisional Patent Application Ser. No. 63/365,406, entitled Polyol Ester of Fatty Acids (PEFAs) as Emulsifiers, filed May 26, 2022; and hereby incorporates the provisional applications by reference herein in their entireties.

### TECHNICAL FIELD

The present disclosure relates to polyol esters of fatty acids (PEFAs) and their use as surfactants, methods of preparing, and compositions thereof.

### BACKGROUND

PEFA compounds can have a variety of uses, including use as surfactants. Surfactants (both petroleum and bio-based) are an example of compounds that can act as emulsifiers, detergents, dispersants, conventional foam control agents, and wetting agents used in a broad variety of consumer applications, including agricultural, nutritional, cosmetic, veterinary, therapeutic, paint, ink, and industrial applications. However, such surfactants can exhibit poor performance with regard to sustainability, bio-accumulation, eco-toxicity and/or biodegradability.

Therefore, there is a need for developing alternative surfactants and compositions that can improve the constitution of consumer products in these applications.

### BRIEF DESCRIPTION OF THE DRAWINGS

FIG. 1 is a chart depicting a critical micelle concentration relative to the surface tension for sodium dodecyl sulfate as a surfactant.

FIG. 2 is a chart depicting a critical micelle concentration relative to the surface tension for a combination of sodium dodecyl sulfate and a PEFA, in a 60:40 ratio, as a surfactant.

### DETAILED DESCRIPTION

Polyol esters of fatty acids are amphiphilic molecules comprising a sugar alcohol, e.g., a D-mannitol and/or a D-arabitol, esterified to the carboxyl end of a 3-hydroxy fatty acyl moiety, which may or may not be acetylated. The non-esterified hydroxy groups of the sugar alcohol may or may not be acetylated as well. In some embodiments, the one or more polyol lipids produced are a mixture of similar compounds containing (R)-3-hydroxy fatty acyl moieties with varying chain lengths, in the range of about 8 to 24 carbons, preferably in the range between 12 to 20 carbons. In some embodiments, the (R)-3-hydroxy fatty acyl moieties can present varying degrees of unsaturation in the range of about 0 to 6, e.g., in the range between 2 to 5 In some embodiments, the non-esterified hydroxy groups of the sugar alcohol can be esterified to acetyl groups. In some embodiments the sugar alcohol can be fully acetylated. In some embodiments, the sugar alcohol can be non-acetylated. In some embodiments, acetylations can range between these two states.

The present disclosure includes, among other things, a compound represented by

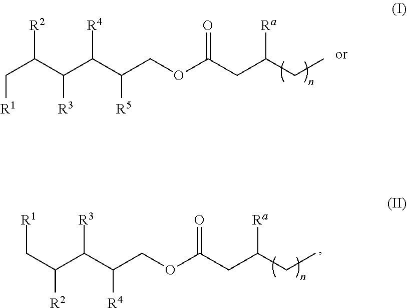

or salt or acid thereof,
wherein R¹, R², R³, R⁴, R⁵, R^{a}, and a are defined herein.

### Definitions

In the present disclosure, the following terms have the following meanings:

The term “about”, preceding a figure encompasses plus or minus 10%, or less, of the value of said figure. It is to be understood that the value to which the term “about” refers is itself also specifically, and preferably, disclosed.

The term “alkyl” refers to a straight, or branched alkyl group. Exemplary alkyl groups are methyl, ethyl, propyl, isopropyl, butyl, isobutyl, and tert-butyt.

The term “halogen” means F, Cl, Br, or I.

As used herein, the term “° surfactant combination” refers to a surfactant comprising PEFA and at least one other surfactant, i.e., a surfactant mixture comprising one or more surfactants formed from PEFA compounds and one or more surfactants formed from non-PEFA compounds.

As used herein, the term “salt or acid thereof” refers to any salts or acids of PEFA compounds. Exemplary salts and acids of PEFA compounds are suitable for use in contact with the tissues of humans and lower animals without undue toxicity, irritation, allergic response, and the like, and are commensurate with a reasonable benefit/risk ratio. Salts can include pharmaceutically or biologically acceptable salts. Likewise, acids can include pharmaceutically or biologically acceptable acids. Pharmaceutically or biologically acceptable salts and acids are well known in the art. For example, S. M. Berge et al., describe pharmaceutically or biologically acceptable salts in detail in J. Pharmaceutical Sciences, 1977, 66, 1-19, incorporated herein by reference. Pharmaceutically or biologically acceptable salts of the compounds of this disclosure can include those derived from suitable inorganic and organic acids and bases, while pharmaceutically or biologically acceptable acids of the compounds of this disclosure can include suitable inorganic and organic acids. Examples of pharmaceutically or biologically acceptable inorganic acids can include hydrochloric acid, hydrobromic acid, phosphoric acid, sulfuric acid, nitric acid and perchloric acid or with organic acids such as acetic acid, oxalic acid, maleic acid, tartaric acid, citric acid, succinic acid, propionic acid, or malonic acid or by using other methods used in the art such as ion exchange. Examples of pharmaceutically or biologically acceptable acid addition salts are salts of an amino group formed with such examples of inorganic acids. Another example of a pharmaceutically or biologically acceptable acid is a carboxylic acid, where the carboxylated version of the PEFA compound includes a carboxyl group. Other pharmaceutically or biologically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically or biologically acceptable salts include, when appropriate, ammonium, quaternary ammonium, and amine cations formed using counterions such as halide, hydroxide, carboxylate, sulfate, phosphate, nitrate, lower alkyl sulfonate and aryl sulfonate.

The present disclosure includes both the (R) and (S) configuration at each stereo center, even in cases where drawn as defined or drawn undefined. Additionally, the present disclosure includes racemic compositions of compounds disclosed herein. The present disclosure includes scalemic compositions of compounds disclosed herein. The present disclosure includes enantioenriched compositions of compounds disclosed herein.

### PEFA Compounds

Disclosed herein are compounds formed from one or more polyol esters of fatty acids (PEFAs) that can provide enhanced benefits to personal care products and other consumer products.

Polyol esters of fatty acids are amphiphilic molecules comprising a sugar alcohol, e.g., a D-mannitol and/or a D-arabitol, esterified to the carboxyl end of a 3-hydroxy fatty acyl moiety, which may or may not be acetylated. The non-esterified hydroxy groups of the sugar alcohol may or may not be acetylated as well. In some embodiments, the one or more polyol lipids produced are a mixture of similar compounds containing (R)-3-hydroxy fatty acyl moieties with varying chain lengths, in the range of about 8 to 24 carbons, preferably in the range between 12 to 20 carbons. In some embodiments, the (R)-3-hydroxy fatty acyl moieties can present varying degrees of unsaturation in the range of about 0 to 6, e.g., in the range between 2 to 5. In some embodiments, the non-esterified hydroxy groups of the sugar alcohol can be esterified to acetyl groups. In some embodiments the sugar alcohol can be fully acetylated. In some embodiments, the sugar alcohol can be non-acetylated. In some embodiments, acetylations can range between these two states.

In some embodiments, a polyol lipid disclosed herein is an acetylated C12:0-3-hydroxy fatty acid esterified to D-arabitol with 3 acetylations. In some embodiments, a polyol lipid disclosed herein is an acetylated C14:0-3-hydroxy fatty acid esterified to D-mannitol with 4 acetylations. In some embodiments, a polyol lipid disclosed herein is an acetylated C16:0-3-hydroxy fatty acid esterified to D-mannitol with 2 acetylations. In some embodiments, a polyol lipid disclosed herein is an acetylated C16:0-3-hydroxy fatty acid esterified to D-arabitol with 2 acetylations. In some embodiments, a polyol lipid disclosed herein is an acetylated C14:0-3-hydroxy fatty acid esterified to D-arabitol with 4 acetylations. In some embodiments, a polyol lipid disclosed herein is an acetylated C14.0-3-hydroxy fatty acid esterified to D-mannitol with 5 acetylations. In some embodiments, a polyol lipid disclosed herein is an acetylated C16:0-3-hydroxy fatty acid esterified to D-mannitol with 3 acetylations. In some embodiments, a polyol lipid disclosed herein is an acetylated C16:0-3-hydroxy fatty acid esterified to D-arabitol with 3 acetylations. In sone embodiments, a polyol lipid disclosed herein is an acetylated C16:0-3-hydroxy fatty acid esterified to D-mannitol with 4 acetylations. In some embodiments, a polyol lipid disclosed herein is an acetylated C18:0-3-hydroxy fatty acid esterified to D-mannitol with 2 acetylations. In some embodiments, a polyol lipid disclosed herein is an acetylated C16:0-3-hydroxy fatty acid esterified to D-mannitol with 5 acetylations. In some embodiments, a polyol lipid disclosed herein is an acetylated C16:0-3-hydroxy fatty acid esterified to D-mannitol with 4 acetylations. In some embodiments, the polyol lipid is an acetylated C18:0-3-hydroxy fatty acid esterified to D-mannitol with 2 acetylations. In some embodiments, a polyol lipid disclosed herein is an acetylated C16:0-3-hydroxy fatty acid esterified to D-mannitol with 5 acetylations. In some embodiments, a polyol lipid disclosed herein is an acetylated C16:0-3-hydroxy fatty acid esterified to D-arabitol with 4 acetylations. In some embodiments, a polyol lipid disclosed herein is an acetylated C1:0-3-hydroxy fatty acid esterified to D-mannitol with 3 acetylations. In some embodiments, a polyol lipid disclosed herein is an acetylated C18:0-3-hydroxy fatty acid esterified to D-mannitol with 4 acetylations. In some embodiments, a polyol lipid disclosed herein is an acetylated C18:0-3-hydroxy fatty acid esterified to D-arabitol with 3 acetylations. In some embodiments, a polyol lipid disclosed herein is an acetylated C18:0-3-hydroxy fatty acid esterified to D-mannitol with 5 acetylations. In some embodiments, a polyol lipid disclosed herein is an acetylated C18:0-3-hydroxy fatty acid esterified to D-arabitol with 4 acetylations. In some embodiments, a polyol lipid disclosed herein is an acetylated C20:0-3-hydroxy fatty acid esterified to D-mannitol with 3 acetylations. In some embodiments, a polyol lipid disclosed herein is an acetylated C20:0-3-hydroxy fatty acid esterified to D-Mannitol with 4 acetylations.

In some example embodiments, a PEFA compound can be represented by:

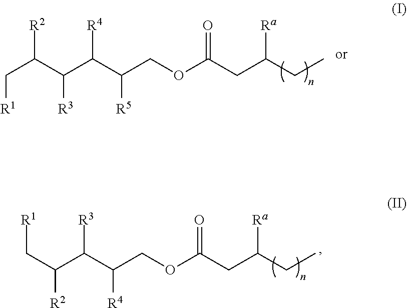

or salt or acid thereof,
wherein
R¹ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —COOH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R² is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼH₂ⱼ₋₁Oⱼ₋₁), —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R³ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R⁴ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R⁵ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R^{a} is selected from the group consisting of —H, —C(O)R^{x}, —R^{x}, —C(O)OR^{y}, —C(O)N(H)R^{y}, (C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
each R^{x} is independently C₁-C₃ alkyl, wherein R^{x} is optionally substituted with 1-7 instances of halogen;
each R^{y} is independently —H, C₁-C₃ alkyl, wherein R^{x} is optionally substituted with 1-7 instances of halogen;
n is 2-20;
j is 2-12;
k is 4-26;
m is 1-16; and
each p is independently selected from 1-10,
wherein
   when n is 12 or 14 and R¹ is —OH or —(O)Ac, then R^{a} is not —(O)Ac or —(O)Me;
   when n is 12 and R^{a} is —OH, then R² is not —OH or;
   when n is 12 or 14, R^{a} is —OH, R² is —OH, R³ is —OH, and R⁴ is —OH, then R¹ is (O)R^{x} or —(O)(C₂H₅O)ₚR^{y};
   when n is 12 or 14, R¹ is —(O)S(O₂)OH, R² is —(O)S(O₂)OH, R³ is —(O)S(O₂)OH, R⁴ is —(O)S(O₂)OH, and R⁵ is —(O)S(O₂)OH, then R^{a} is —(O)C(O)R^{x}, R^{x}, —(O)C(O)OR^{x}, —(O)C(O)N(H)R^{x}, —(O)(C₂H₅O)ₚR^{y}, —(O)P(O)(OH)₂, or —(O)S(O₂)OH.

In some embodiments with respect to Formula (I) or (II),
R¹ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —COOH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R² is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R³ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R⁴ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R⁵ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R^{a} is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
each R^{x} is independently C₁-C₃ alkyl, wherein R^{x} is optionally substituted with 1-7 instances of halogen;
each R^{y} is independently —H, C₁-C₃ alkyl, wherein R^{x} is optionally substituted with 1-7 instances of halogen;
n is 2-11;
j is 2-12;
k is 4-26;
m is 1-16; and
each p is independently selected from 1-10.

In some embodiments, a PEFA compound can further be represented by Formula (I-a) or (II-a):

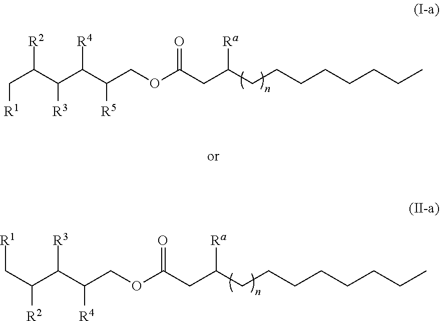

or salt or acid thereof,
wherein
n is 6-12.

In some embodiments, R¹ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —COOH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚRₓ, —(O)P(O)(OH)₂, —(O)S(O₂)OH, —(O)NO₂, —(O)NH₂, —(O) (CⱼHₖOₘ). In some embodiments, R^{x} is selected from the group consisting of —C(C))R^{x}, —R^{x}—, —C(O)OR^{x}, —C(O)N(H)R^{y}, —(C₂H₅O)ₚR^{y}, —P(O)(O)H)₂, and —S(O₂)OH. In some embodiments, R¹ is —H. In some embodiments, R¹ is —P(O)(OH)₂, or —S(O₂)OH. In some embodiments, R¹ is —C(O)R^{x}. In some embodiments, R¹ is —Ac. In some embodiments, R¹ is —R^{x}. In some embodiments, R¹ is —C(O)OW. In some embodiments, R¹ is —C(O)N(H)R^{x}. In some embodiments, R¹ is —(C₂H)ₚR^{y}. In some embodiments, R¹ is —P(O)(OH)₂. In some embodiments, R¹ is —S(O₂)OH. In some embodiments, R¹ is —(CⱼHₖOₘ).

In some embodiments, R² is selected from the group consisting of —H, —C(O)R^{x}—, —R^{x}, C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ). In some embodiments, R² is selected from the group consisting of —C(O)R^{x}, —R^{x}, —C(O)OR^{x}, —C(O)N(H)R^{y}, —(C₂H₅O)ₚR^{y}, —P(O)(OH)₂, and —S(O₂)OH. In some embodiments. R² is —H. In some embodiments, R² is —P(O)(OH)₂, or —S(O₂)OH. In some embodiments, R² is —C(O)R^{x}. In some embodiments, R² is —Ac. In some embodiments, R² is —R^{x}. In some embodiments, R² is —C(O)OR³. In some embodiments, R² is —C(O)N(H)R^{y}. In some embodiments, R² is —(C₂H₅O)ₚR^{y}. In some embodiments, R² is —P(O)(OH)₂. In some embodiments, R² is —S(O₂)OH. In some embodiments. R² is —(CⱼHₖOₘ). In some embodiments, R³ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}—, —(O)P(O)(OH)₂, —(O)S(O₂)OH, —(O)NO₂, —(O)NH₂, —(O)(CⱼHₖOₘ). In some embodiments, R³ is selected from the group consisting of —C(O)R′, —R^{x}, —C(O)OR^{y}, —C(O)N(H)R^{y}, —(C₂H₅O)ₚR^{y}, —P(O)(OH)₂, and —S(O₂)OH. In some embodiments, R³ is —H. In some embodiments, R³ is —P(O)(OH)₂, or —S(O₂)OH. In some embodiments, R³ is —C(O)R^{x}. In some embodiments, R³ is —Ac Tn some embodiments, R³ is —R^{x}. In some embodiments, R³ is —C(O)OR^{y}. In some embodiments, R³ is —C(O)N(H)R^{y}. In some embodiments, R³ is —(C₂H₅O)ₚR^{y}. In some embodiments, R³ is —P(O)(OH)₂. In some embodiments, R³ is —S(O₂)OH. In some embodiments, R³ is —(CⱼHₖOₘ). In some embodiments, R⁴ is selected from the group consisting of —H₁, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ). In some embodiments, R⁴ is selected from the group consisting of —C(O)R^{x}, —R^{x}, —C(O)OR^{y}, —C(O)N(H)R^{y}, —(C₂H₅O)ₚR^{y}, —P(O)(OH)₂, and —S(O₂)OH—In some embodiments, R⁴ is —H. In some embodiments, R⁴ is —P(O)(OH)₂, or —S(O₂)OH. In some embodiments, R⁴ is —C(O)R^{x}. In some embodiments, R⁴ is —Ac. In some embodiments, R⁴ is —R^{x}. In some embodiments, R⁴ is —C(O)OR^{y}. In some embodiments, R⁴ is —C(O)N(H)R^{y}. In some embodiments, R⁴ is —(C₂H₂O)ₚR^{y}. In some embodiments, R⁴ is —P(O)(OH)₂. In some embodiments, R⁴ is —S(O₂)OH. In some embodiments, R⁴ is —(CⱼHₖOₘ). In some embodiments, R⁵ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ). In some embodiments, R⁵ is selected from the group consisting of —C(O)Rₓ, —R^{x}, —C(O)OR^{y}, —C(O)N(H)R^{y}, —(C₂H₅O)ₚR^{y}, —P(O)(OH)₂, and —S(O₂)OH. In some embodiments, R⁵ is —H. In some embodiments, R⁵ is —P(O)(OH)₂, or —S(O₂)OH. In some embodiments, R⁵ is —C(O)R^{x}. In some embodiments, R⁵ is —Ac. In some embodiments, R⁵ is —R^{x}. In some embodiments, R⁵ is —C(O)OR^{y}. In some embodiments, R⁵ is —C(O)N(H)R^{y}. In some embodiments, R⁵ is —(C₂H₅O)ₚR^{y}. In some embodiments, R⁵ is —P(O)(OH)₂. In some embodiments, R⁵ is —S(O₂)OH. In some embodiments, R⁵ is —(CⱼHₖOₘ). In some embodiments, R^{a} is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ). In some embodiments, R^{a} is selected from the group consisting of —C(O)R^{x}, R^{x}, —C(O)OR^{y}, —C(O)N(H)R^{y}, —(C₂H₅O)ₚR^{y}, —P(O)(OH)₂, and —S(O₂)OH. In some embodiments, R^{a} is —H. In some embodiments, R^{a} is —P(O)(OH)₂ or —S(O₂)OH. In some embodiments, R^{a} is —C(C)R^{x} In some embodiments, R^{a} is —Ac. In some embodiments, R^{a} is —R^{x}. In some embodiments, R^{a} is —C(O)OR^{y}. In some embodiments, R^{a} is —C(O)N(H)R^{y}. In some embodiments, R^{a} is —(C₂H₅O)ₚR^{y}. In some embodiments, R^{a} is —P(O)(OH)₂. In some embodiments, R^{a} is —S(O₂)OH. In some embodiments, R^{a} is —(CⱼHₖOₘ).

In some embodiments, each R^{x} is independently C₁-C₃ alkyl, wherein R^{x} is optionally substituted with 1-7 instances of halogen. In some embodiments, R^{x} is methyl, wherein R^{x} is optionally substituted with 1-7 instances of halogen. In some embodiments, R^{x} is ethyl, wherein R^{x} is optionally substituted with 1-7 instances of halogen. In some embodiments, R^{x} is n-propyl, wherein R^{x} is optionally substituted with 1-7 instances of halogen. In some embodiments, R^{x} is i-propyl, wherein R^{x} is optionally substituted with 1-7 instances of halogen. In some embodiments, R^{x} is methyl. In some embodiments, R^{x} is ethyl. In some embodiments, R^{x} is n-propyl. In some embodiments, R^{x} is i-propyl.

In some embodiments, each RN is independently —H or C₁-C₃ alkyl, wherein R^{y} is optionally substituted with 1-7 instances of halogen. In some embodiments, R^{y} is —H. In some embodiments, each R^{y} is independently C₁-C₃ alkyl, wherein R^{y} is optionally substituted with 1-7 instances of halogen. In some embodiments, R^{y} is methyl, wherein R^{y} is optionally substituted with 1-7 instances of halogen. In some embodiments, R^{y} is ethyl, wherein R^{y} is optionally substituted with 1-7 instances of halogen. In some embodiments, R^{y} is n-propyl, wherein R^{y} is optionally substituted with 1-7 instances of halogen. In some embodiments, R^{y} is i-propyl, wherein R^{y} is optionally substituted with 1-7 instances of halogen. In some embodiments, R^{y} is methyl. In some embodinents, R^{y}, is ethyl. In sone embodiments, R^{y} is n-propyl. In some embodiments, R^{y} is i-propyl.

The present disclosure includes PEFA compounds represented by:

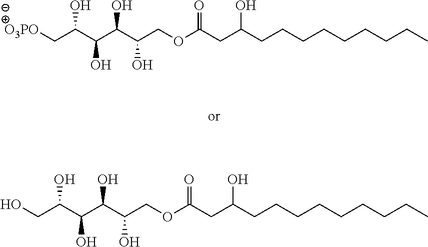

or sat or acid thereof.

Below are examples of specific embodiments regarding the invention described by the present disclosure. The examples are offered for illustrative purposes only and are not intended to limit the scope of the present invention in any way. The groups referenced in the examples below, e.g., OR¹, OR², etc., refer to the corresponding groups described herein, e.g., R¹, R², etc., and can be replaced with any of the groups provided in the respective corresponding group, i.e., without the “O.” Efforts have been made to ensure accuracy with respect to numbers used (e.g., amounts, temperatures, etc.), but some experimental error and deviation should, of course, be allowed for.

In some embodiments, compounds of the present disclosure can be prepared as outlined in Scheme 1 or 2:

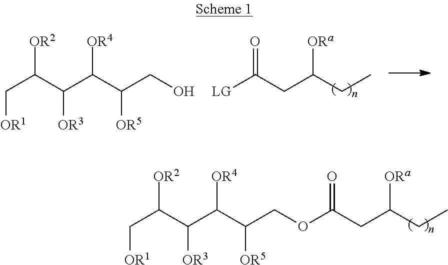

wherein LG is a leaving group.

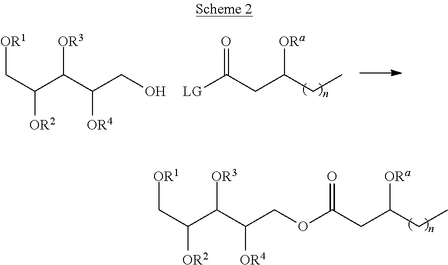

wherein LG is a leaving group.

In some embodiments compounds of the present disclosure can be prepared as outlined in Scheme 3 or 4:

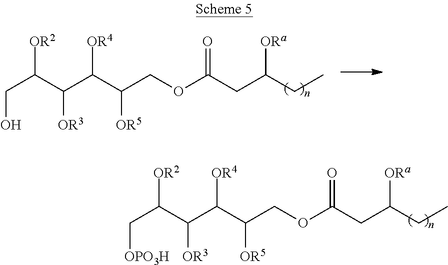

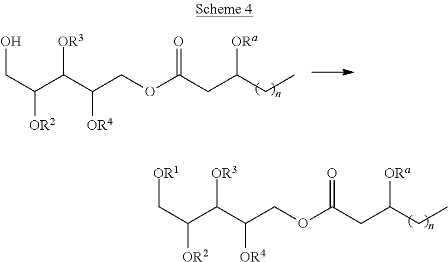

In some embodiments, compounds of the present disclosure can be prepared as outlined in Scheme 5 or 6:

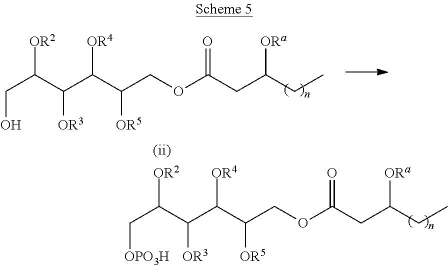

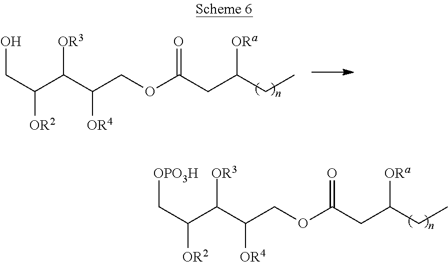

Synthesis/isolation of starting materials for the compounds described herein can be found in WO2018148465 and WO2017184884, each of which are incorporated in their entirety. One example for synthesizing 3-Hydroxy-dodecanoic acid 2,3,4,5-tetrahydroxy-6-phosphonooxy-hexyl ester is provided below.

### Synthesis of 3-Hydroxy-dodecanoic acid 2,3,4,5-tetrahydroxy-6-phosphonooxy-hexyl ester

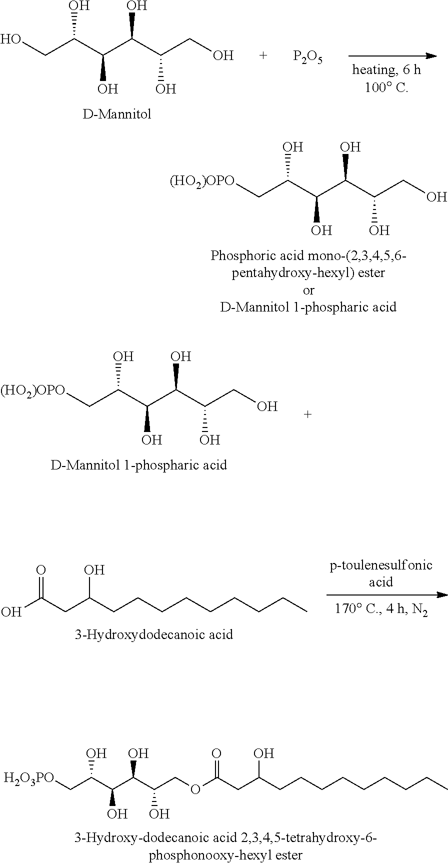

D-mannitol and P₂O₅ are heated neat at 100° C. for 6 hours. As an example, D-Mannitol 1-phosphoric acid can be prepared as outlined in J.-G. Nam et al. Materials Chemistry and Physics 116 (2009) 46-51. D-Mannitol 1-phosphoric acid and 3-hydroxydodecanoic acid are heated in p-toluene sulfonic acid at 170° C. for 4 h at N₂ as outlined in Wenyuan Han et al. Polymers 11, (2019), 1031.

### Surfactants

Surfactants are interfacially active compounds They generally have a polar head group and a non-polar hydrocarbon chain. The polar part of the molecule can interact with polar solvents, like water, and is therefore also called the hydrophilic part. The non-polar part, on the other hand, can form interactions with non-polar solvents, like oil, and is therefore also called lipophilic or hydrophobic part. There are four basic classes of surfactants: anionic surfactants, cationic surfactants, zwitterionic surfactants, and nonionic surfactants.

Surfactants are amphiphilic molecules that are widely used in consumer products, industrial processes, and biological applications. A critical property of a surfactant is the critical micelle concentration (CMC), which is the concentration at which the surfactant molecules start to assemble into clusters. Micelles consist of agglomerates of surfactant molecules inside the liquid and facilitate washing by storing hydrophobic substances (fats, oils, etc.) within the agglomerates. The CMC value is defined as the concentration of surfactants above which micelles start to form and the surface tension of the formulation remains relatively constant. The CMC value indicates the amount of surfactant required to reach maximum surface tension reduction. The lower the CMC value, the less surfactant is required to effectively emulsify, solubilize, and disperse hydrophobic substances in the formulation. Among the surfactant classes—nonionic, anionic, cationic and amphoteric—the lowest CMC's are generally found in the nonionic category. Typically, nonionic surfactants with very low CMC values can deliver excellent wetting and detergency benefits. Thus, CMC values measure the efficiency of surfactants, and those with lower values will exhibit desired benefits when used in consumer products, industrial processes, and biological applications.

These CMC values can be determined for surfactant solutions by measuring the surface tension at different concentrations. CMC values can be calculated from different known techniques (e.g., tensiometry, conductivity, fluorescence spectroscopy). In some embodiments, the PEFA surfactants described herein in combination with another surfactant (surfactant combination) can have a (CMC value of about 0.1 mM or less; in some embodiments a. CIC value of about 0.07 mM or less; in some embodiments a CMC value of about 0.05 mM or less; in some embodiments a CMC value of about 0.04 mM or less; in some embodiments a CMC value of about 0.03 mM or less. In some embodiments, such surfactant combinations can have a CMC value from about 0.01 nM to about 0.1 mM; and in some embodiments, a CMC value from about 0.01 to about 0.05 mM. In certain surfactant combinations, the presence of the PEFA compounds described herein and a second surfactant can substantially lower the amount of the second surfactant (e.g., sodium dodecyl sulfate, SDS) needed to achieve a desired surface tension. For example, the amount of SDS and/or sodium dodecyl benzenesulfonate (SDBS) needed to achieve a desired surface tension of a formulation can be lowered by over 10 times, over 50 times or over 100 times with the addition of a PEFA surfactant as described herein, thus rendering the surfactant mixture more beneficial and effective.

Surfactants can contain one or more PEFA compounds. In some embodiments, a surfactant can have pentitol polyol esters of fatty acids and hexitol polyol esters of fatty acids. In some embodiments, the C16 and C18 fatty acid components of the pentitol and hexitol polyol esters can comprise about 20% to about 99% of the PEFA; and in some embodiments from about 30% to about 95% of the PEFA; in some embodiments from about 40% to about 90% of the PEFA In some embodiments, the C16 and C18 fatty acid components of the pentitol and hexitol polyol esters can comprise about 50% or greater of the PEFA; in some embodiment, about 60% or greater of the PEFA; and in some embodiments, about 70% or greater of the PEFA; and in some embodiments, about 80% or greater of the PEFA. In some embodiments, the ratio of pentitol to hexitol polyol esters in the surfactant is about 5:1 or greater; in some embodiment, the ratio is about 8:1 or greater; in some embodiments, the ratio is about 10:1 or greater; in some embodiments, the ratio is about 15:1 or greater; in some embodiments is about 20:1 or greater; in some embodiments, the ratio is about 30:1 or greater; in some embodiments, the ratio is about 40:1 or greater; in some embodiments is about 50:1 or greater.

The surfactants described herein can have a wide array of applications. For example, such surfactants could be included in personal care compositions. Some examples of such personal care compositions include, skin moisturizers, perfumes, lipsticks, fingernail polishes, eye and facial makeup preparations, shampoos, permanent waves, hair colors, toothpastes, and deodorants. The surfactants could be used in cleaning compositions. Some examples of such cleaning compositions include hard surface cleaners, all-purpose cleaners, bleaches, detergents, and sanitizers. The surfactants described herein would be suitable for any personal care composition or cleaning composition where an effective surfactant is needed.

### Examples

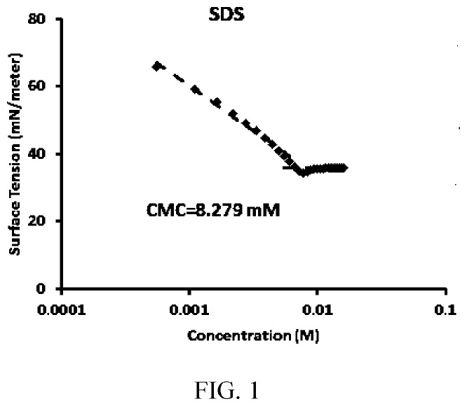

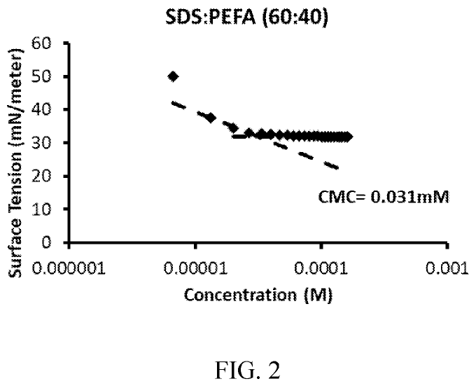

CMC values were measured for water-soluble surfactants at different ratios for a mixture of surfactants. CMC values were calculated using known techniques utilizing plots of surface tension versus the logarithm of varying surfactant concentrations. The measured CMC values of PEFA in combination with sodium dodecyl sulfate (SDS) at a 60:40 (weight:weight) ratio, CMC values of other nonionic surfactants and PEFA at the same ratios are shown in Table 1. FIGS. 1 and 2 show representative CMC measurements obtained from surfactants comprising SDS and PEFA at the indicated weight ratios. As shown in FIG. 2, the surfactant combination of SDS and PEFA at a 60:40 ratio have the lowest CMC value (0.031 mM) in comparison to all other reference non-ionic surfactants. Table 1 discloses that the CMC values of neat SDS and neat SDBS are 8.2 mM and 2.5 mM, respectively. In addition, PEFA is soluble in SDBS at 60:40 unlike cetearyl glucoside. Table 1 also discloses that a mixture of SDBS and PEFA at a 60:40 ratio was measured, and had a CMC value of 0.22 mM an order of magnitude lower than all other reference surfactants. The CMC values of a surfactant combination of SDBS and CGlucoside at 80:20 could not be measured because CGlucoside is insoluble in SDBS. Thus, PEFA compounds in combination with other ionic surfactants have high efficiency due to the low CMC values of the combination of ionic surfactants, such as SDS and SDBS, with PEFA.

TABLE 1

| | CMC | | CMC | | CMC |
|---|---|---|---|---|---|
| Surfactant | (mM) | Surfactant | (mM) | Surfactant | (mM) |
| SDS | 8.2 | SDS:PEFA | 0.058 | SDBS | 2.5 |
| | | (70:30) | | | |
| SDS:PEFA | 0.068 | SDS:PEFA | 0.031 | SDBS:PEFA | 0.022 |
| (80:20) | | (60:40) | | (80:20) | |
| SDS:NOVEL 30 | 0.107 | SDS:NOVEL 30 | 0.14 | SDBS:NOVEL 30 | 0.19 |
| (80:20) | | (60:40) | | (80:20) | |
| SDS:Sophorolipid | 0.19 | SDS:Sophorolipid | 0.138 | SDBS:Sophorolipid | 0.29 |
| (80:20) | | (60:40) | | (80:20) | |
| SDS:SPAN 40 | 0.75 | SDS:SPAN 40 | Insoluble | SDBS:SPAN 40 | 0.32 |
| (80:20) | | (60:40) | | (80:20) | |
| SDS:CGlucoside | 1.78 | SDS:CGlucoside | Insoluble | SDBS:CGlucoside | Insoluble |
| (80:20) | | (60:40) | | (80:20) | |

Those skilled in the art will recognize or be able to ascertain using 10n more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A PEFA compound represented by Formula (I) or (II): 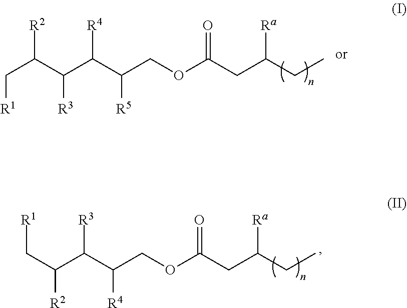
or salt or acid thereof,
wherein
R¹ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —COOH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R² is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R³ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R⁴ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R⁵ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, −(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R^{a} is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
each R^{x} is independently C₁-C₃ alkyl, wherein R^{x} is optionally substituted with 1-7 instances of halogen;
each R^{y} is independently —H, or C₁-C₃ alkyl, wherein R^{x} is optionally substituted with 1-7 instances of halogen;
n is 2-20;
j is 2-12;
k is 4-26;
m is 1-16; and
each p is independently selected from 1-10,
wherein
when n is 12 or 14 and R¹ is —OH or —(O)Ac, then R^{a} is not —(O)Ac or —(O)Me;
when n is 12 and R^{a} is —OH, then R² is not —OH or;
when n is 12 or 14, R^{a} is —OH, R² is OH, R³ is OH, and R⁴ is OH, then R¹ is (O)R^{x} or —(O)(C₂H₅O)ₚR^{y};
when n is 12 or 14, R¹ is —(O)S(O₂)OH, R² is —(O)S(O₂)OH, R³ is —(O)S(O₂)OH, R⁴ is —(O)S(O₂)OH, and R⁵ is —(O)S(O₂)OH, then R^{a} is —(O)C(O)R^{x}, R^{x}, —(O)C(O)OR^{x}, —(O)C(O)N(H)R^{x}, —(O)(C₂H₅O)ₚR^{y}, —(O)P(O)(OH)₂, or —(O)S(O₂)OH.

2. The PEFA compound of claim 1, wherein
R¹ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —COOH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R² is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R³ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R⁴ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R⁵ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R^{a} is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
each R^{x} is independently C₁-C₃ alkyl, wherein R^{x} is optionally substituted with 1-7 instances of halogen;
each R^{y} is independently —H, or C₁-C₃ alkyl, wherein R^{x} is optionally substituted with 1-7 instances of halogen;
n is 4-12;
j is 2-12;
k is 4-26;
m is 1-16; and
each p is independently selected from 1-10.

3. The PEFA compound of claim 1, wherein n is 4-20.

4. The PEFA compound of any of claims 1-2, wherein R¹ is —(O)P(O)(OH)₂.

5. The PEFA compound of any of claims 1-2, wherein R¹ is —(O)Ac.

6. The PEFA compound of any of claims 1-2, wherein R¹ is —(O)S(O₂)OH.

7. The PEFA compound of any of claims 1-2, wherein R¹ is —OH.

8. The PEFA compound of any of claims 1-7, wherein R^{a} is —OH.

9. The PEFA compound of any of claims 1-7, wherein R^{a} is —(O)Me.

10. The PEFA compound of any of claims 1-7, wherein R^{a} is —(O)Ac.

11. The PEFA compound of any of claims 1-10, wherein n is 7-9.

12. The PEFA compound of any of claims 1-10, wherein n is 8.

13. The PEFA compound of any of claims 4-10, wherein the compound is further represented by Formula (I-a) or (II-a): 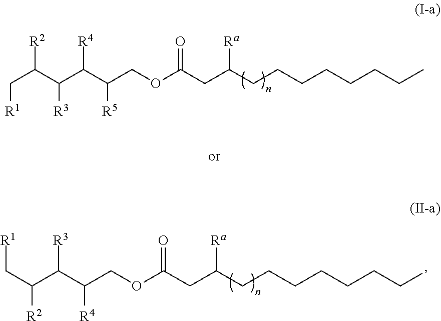
or salt or acid thereof,
wherein
n is 6-12.

14. The PEFA compound of claim 13, wherein n is 7-9.

15. The PEFA compound of claim 13, wherein n is 8.

16. The PEFA compound of claim 13, wherein n is 6.

17. A PEFA compound selected from the group consisting of: 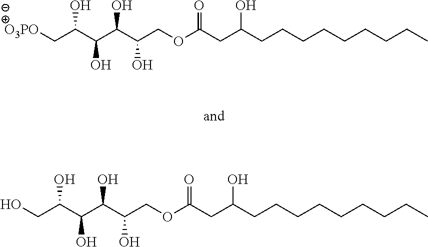
or salt or acid thereof.

18. A surfactant formed from one or more PEFA compounds of any of claims 1-17, wherein the surfactant has a CMC value of about 0.09 mM or less.

19. The surfactant of claim 18 having a CMC value of about 0.05 mM or less.

20. The surfactant of claim 18 having a CMC value of about 0.03 mM or less.

21. The surfactant of claim 18 having a CMC value from about 0.01 mM to about 0.09 mM.

22. The surfactant of claim 18 having a CMC value from about 0.01 mM to about 0.5 mM.

23. The surfactant of claim 18, wherein the one or more PEFA compounds comprise pentitol polyol esters, hexitol polyol esters, or a combination thereof.

24. The surfactant of claim 23, wherein the ratio of pentitol to hexitol polyol esters is about 5:1 or greater.

26. The surfactant of claim 23, wherein the ratio of pentitol to hexitol polyol esters is about 20:1 or greater.

27. The surfactant of claim 23, wherein the C16 and C18 fatty acid components of the pentitol and hexitol polyol esters can comprise about 20% to about 99% of the PEFA.

28. The surfactant of claim 23, wherein the C16 and C18 fatty acid components of the pentitol and hexitol polyol esters can comprise about 30% to about 95% of the PEFA.

29. The surfactant of claim 23, wherein the C16 and C18 fatty acid components of the pentitol and hexitol polyol esters can comprise about 40% or more of the PEFA.

30. A surfactant composition comprising the surfactant of claims 18-28 and one or more surfactants formed from non-PEFA compounds.

31. A personal care composition comprising the surfactant of claims 18-28.

32. A cleaning composition comprising the surfactant of claims 18-22.
